(19) United States
(12) Patent Application Publication
(10) Pub. No.: US 2024/0428889 A1
(43) Pub. Date: Dec. 26, 2024

(54) System and Method for Expression Amounts of DNA Sequences in a Biological Sample

(71) Applicant: Echelon Diagnostics, Inc., Reno, NV (US)

(72) Inventors: John BURKE, Reno, NV (US); Stephen Healy Sanders, Reno, NV (US)

(21) Appl. No.: 18/830,129

(22) Filed: Sep. 10, 2024

Related U.S. Application Data
(63) Continuation of application No. 15/773,238, filed on May 3, 2018, now abandoned.
(60) Provisional application No. 62/250,108, filed on Nov. 3, 2015.

(51) Int. Cl. G16B 30/00; G06N 7/08; G06N 20/00; G16B 20/00; G16B 99/00

(52) U.S. Cl. CPC G16B 30/00; G06N 7/08; G16B 99/00; G01N 2800/385; G06N 20/00; G16B 20/00

(57) ABSTRACT

Some techniques for compensating noise in nucleotide sequencing data include smoothing data for a first reference sequence based on amounts of a subset of reference sequences. An amount of each reference sequence of the subset is multiplied by a corresponding smoothing factor for the reference sequence. The smoothing factor for the reference sequence is based on a spread of the amounts of the reference sequence in the training data. Some techniques include applying a hidden Markov model in which hidden states represent normal condition and a condition of interest at each of multiple pairs of complementary fractions of the sample exhibiting the condition. Transitions from a non-normal condition at a first fraction are confined to states at the first fraction. The normal condition at the first fraction can transition to a state of a different fraction only if the different state represents the normal condition at a complementary fraction.

### CROSS-REFERENCE TO RELATED APPLICATIONS

This application is a continuation under 35 U.S.C. § 120 of U.S. patent application Ser. No. 15/773,238 filed May 3, 2018, which is a 371 National Stage application of PCT Application No. PCT/US16/60270 filed Nov. 3, 2016, which claims benefit of U.S. Provisional Appln. 62/250,108, filed Nov. 3, 2015, under 35 U.S.C. § 119 (c), the entire contents of which are hereby incorporated by reference as if fully set forth herein

### BACKGROUND

Massively Parallel Sequencing (MPS) approaches such as those now in wide commercial use (Illumina/Solexa, Roche/454 Pyrosequencing, and ABI SOLID) are attractive tools for sequencing. Typically, MPS methods can only obtain short read lengths (hundreds of base pairs, bp, also called nucleotides, nt, with Illumina platforms, to a maximum of 200-300 nt by 454 Pyrosequencing) but perform many thousands to millions of such short reads on the order of hours. Sanger methods, on the other hand, achieve longer read lengths of approximately 800 nt (typically 500-600 nt with non-enriched DNA) but take several times longer to do so.

While sequencing machines were originally created for the purposes of sequencing genomic DNA, they have since been put to a myriad of other uses. Considering a sequencer simply as a device for recording the count of specific DNA sequences, sequence census experiments utilize high-throughput sequencing to estimate abundances of “target sequences” (also called “reference sequences”) for molecular biology and biomedical applications. Unusual populations of certain reference sequences can be diagnostic of disease.

To compare the DNA of the sequenced sample to its reference sequence, current methods are designed to find the corresponding part of that sequence for each read in the output sequencing data. This step is called aligning or mapping the reads against the reference sequence. Once this is done, one can look for one or more variations (e.g., a single nucleotide polymorphism, SNP, or a copy number variation, CNV, or a structural variation like presence/absence variation, PAV, or multiples or combinations thereof) within the sample. Aligning the read to the reference consumes a considerable amount of computing power.

For example, Sehnert et al 2011 and Biananchi et al 2014 describe methods to identify aneuploidy in a fetus from maternal blood samples, thus avoiding expensive and dangerous invasive procedures. Aneuploidy is a condition in which the number of chromosomes in the nucleus of a cell is not an exact multiple of the monoploid number of a particular species. In humans, the normal cell has two copies of each chromosome, called diploid, while an aberrant cell might have fewer copies (0 called a deletion, 1 called monosomy) or more copies (3 called trisomy, etc.). An extra or missing chromosome, or a significant portion thereof, is a common cause of genetic disorders including human birth defects. The fetal DNA in maternal blood is a very small fraction of the sample (e.g., less than 10% and often as little as 0.5%) and the identification of its sequences is thus subject to systematic and random errors in the sample preparation, sequencing and alignment processes.

Similarly, cancerous tumors may have CNVs, presence absence variations (PAVs), other structural mutations, or express different genes than the populations of normal cells in an individual. The tumor DNA in a patient tissue sample is a relatively small fraction of the sample (e.g., less than 15% and sometimes as little as 0.5%) and the identification of its sequences is likewise subject to systematic bias and random errors in the sample preparation, sequencing and alignment processes.

### SUMMARY

Techniques are provided for improved accuracy and sensitivity of DNA testing by reducing or otherwise compensating for random noise.

In a first set of embodiments, a method includes obtaining on a processor first data that indicates an amount of each of multiple reference sequences for nucleic acids from each of multiple training samples. The reference sequences include a target reference sequence for which a relative abundance compared to other reference sequences is indicative of a condition of interest. Multiple spreads of the amounts corresponding to the plurality of reference sequences are determining automatically on the processor. Each spread, corresponding to the amounts of each reference sequence, is based on a measure of spread in a portion of the first data for the particular reference sequence among the training samples. The method includes obtaining on a processor second data that indicates an amount of each of the reference sequences from a sample from a subject, wherein the sample is different from the training samples. The method still further includes determining automatically on a processor smoothed second data that indicates a smoothed amount of a first reference sequence based on a portion of the second data for a subset of the reference sequences. An amount of each reference sequence of the subset is multiplied by a corresponding smoothing factor for the reference sequence. The smoothing factor for the reference sequence is based on the spread of the amounts of the reference sequence in the training data. The method yet further includes presenting on a display output data related to the condition of interest in the subject based on the smoothed second data.

In a second set of embodiments, a method includes obtaining on a processor first data that indicates an amount of each of multiple reference sequences for nucleic acids from a sample from a subject. The reference sequences include a target reference sequence for which a relative abundance compared to other reference sequences is indicative of a condition of interest. The method also includes determining automatically on the processor expected values for multiple hidden states of a hidden Markov model based on the first data. The method still further includes presenting on a display output data related to the condition of interest in the subject based on the expected values for the plurality of hidden states of the hidden Markov model. The hidden states represent a plurality of conditions, including one normal condition and the condition of interest, at each of multiple pairs of complementary fractions, each indicating a fraction of the sample exhibiting one of the conditions. A fraction complementary to a different fraction is equal to one minus the different fraction. Transitions from a first hidden state representing a condition different from the normal condition at a first fraction are confined to transitions to hidden states at the first fraction. A hidden state representing the normal condition at the first fraction can transition to a different hidden state representing a different fraction only if the different hidden state represents the normal condition at a complementary fraction.

In other sets of embodiments, a computer-readable medium, a system, or an apparatus is configured to cause an apparatus to perform one or more steps of the above methods.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1A through FIG. 1C are block diagrams that illustrate relative abundance of reference sequences in a sample;

FIG. 1D is a block diagram that illustrates an example process to obtain reads from a sample and associate reads with reference sequences, according to an embodiment;

FIG. 2 is a flow chart that illustrates a method for compensating for noise to increase the accuracy and sensitivity of DNA testing, according to an embodiment;

FIG. 3A and FIG. 3B are graphs of successive portions of a plot that illustrates example distribution of abundances of regions of a chromosome among thousands of reads during processing, according to an embodiment;

FIG. 4 is a block diagram that illustrates features of a new hidden Markov model (HMM) that compensates for noise in samples with contributions from a small fraction, according to an embodiment;

FIG. 5 is a graph that illustrates model solution and corresponding copy number for portions of a chromosome, according to an embodiment.

FIG. 6 is a block diagram that illustrates a computer system upon which an embodiment of the invention may be implemented; and

FIG. 7 is a block diagram that illustrates a chip set upon which an embodiment of the invention may be implemented.

### DETAILED DESCRIPTION

A method and apparatus are described for compensating for noise in order to provide improved accuracy and sensitivity of DNA testing. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus a value 1.1 implies a value from 1.05 to 1.15. The term “about” is used to indicate a broader range centered on the given value, and unless otherwise clear from the context implies a broader rang around the least significant digit, such as “about 1.1” implies a range from 1.0 to 1.2. If the least significant digit is unclear, then the term “about” implies a factor of two, e.g., “about X” implies a value in the range from 0.5× to 2×, for example, about 100 implies a value in a range from 50 to 200. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

Some embodiments of the invention are described below in the context of identifying aneuploidy in a fetus from a maternal blood sample. However, the invention is not limited to this context. In other embodiments, the nucleotide sequencing and imposed model (or smoothing or both) are used to detect other genetic defects, detect the presence of tumors (e.g., by analysis of circulating tumor DNA in blood), classify differences among different samples, or provide a census of expressed genes. In some embodiments, the method is applied to plant and animal screening for the identification of aneuploidy, chromosomal defects, as well as identification of translocations. The ability to detect signal present at low fraction of the total signal also is applicable to the surveillance and detection of unlicensed use of crop and seed germplasm. For example, in a bag of seed, only one out of several hundred seeds may contain protected inbred seed.

### 1. OVERVIEW

Deoxyribonucleic acid (DNA) is a, usually double-stranded, long molecule that is used by biological cells to encode other shorter molecules, such as proteins, used to build and control all living organisms. DNA is composed of repeating chemical units known as “nucleotides” or “bases.” There are four bases: adenine, thymine, cytosine, and guanine, represented by the letters A, T, C and G, respectively. Adenine on one strand of DNA always binds to thymine on the other strand of DNA; and guanine on one strand always binds to cytosine on the other strand and such bonds are called base pairs. Any order of A, T, C and G is allowed on one strand, and that order determines the reverse complementary order on the other strand. The actual order determines the function of that portion of the DNA molecule. Information on a portion of one strand of DNA can be captured by ribonucleic acid (RNA) that also is composed of a chain of nucleotides in which uracil (U) replaces thymine (T). Determining the order, or sequence, of bases on one strand of DNA or RNA is called sequencing. A portion of length k bases of a strand is called a k-mer; and specific short k-mers are called oligonucleotides or oligomers or “oligos” for short.

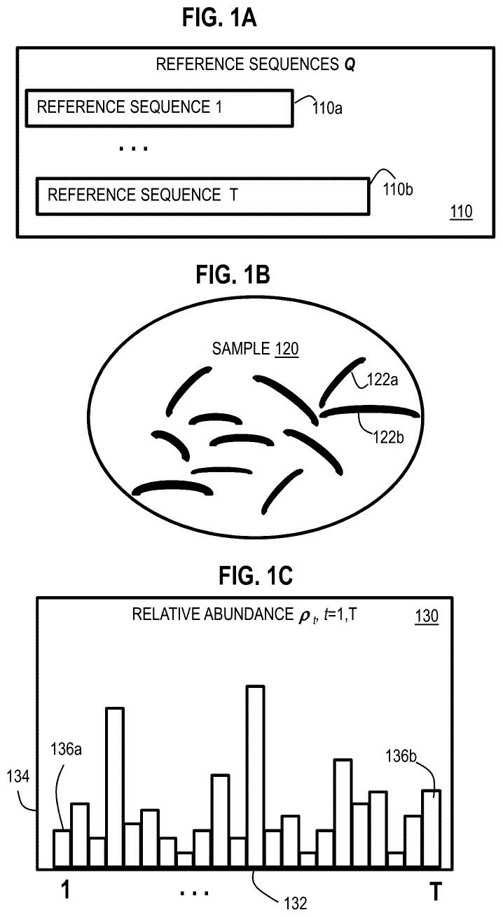

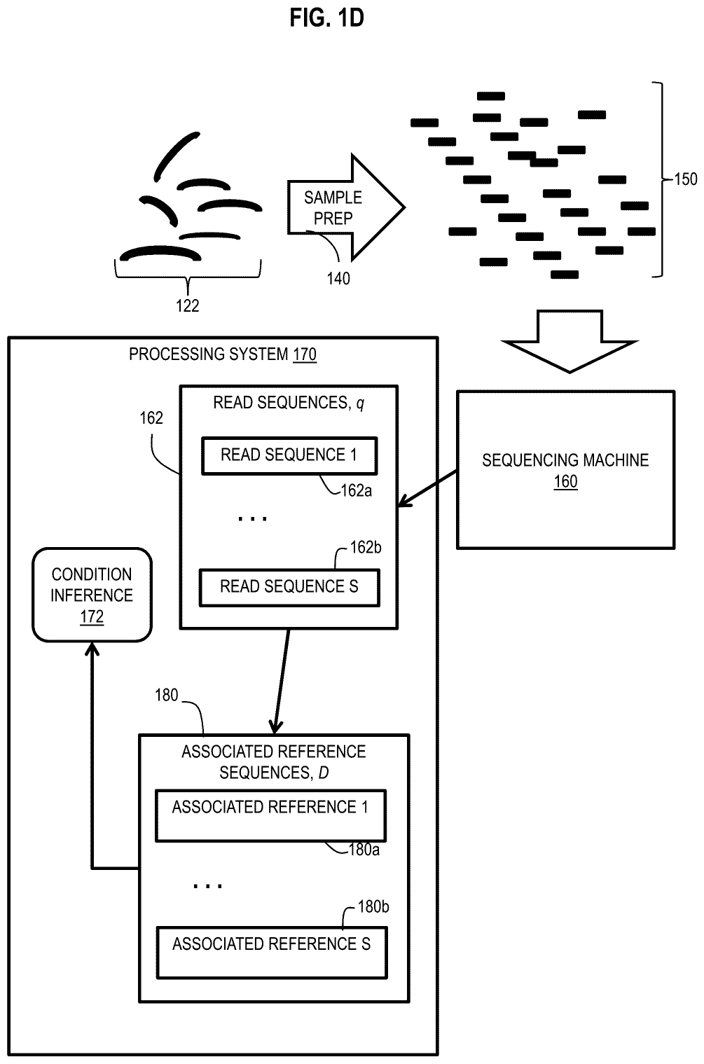

FIG. 1A through FIG. 1C are block diagrams that illustrate relative abundance of reference sequences in a sample. FIG. 1A is a block diagram that illustrates an example data structure 110 of C reference sequences Q, including field 110a holding data that indicates first reference sequence (Q₁), through field 110b holding the last (Tth) reference sequence (Q_{T}), among others indicated by ellipsis. An individual reference sequence is indicated by Qₜ, where t∈{1, . . . , T}. A reference sequence can refer to a normal (also called most common or consensus sequence or baseline or disease free sequence) or a SNP, CNV, PAV or other known structural variation of the normal sequence.

FIG. 1B is a block diagram that represents an example sample 120 with multiple occurrences of nucleic acids, e.g., 122a, 122b (collectively referenced hereinafter as nucleic acids 122) each having at least one of the reference sequences. There may be several occurrences of a nucleic acid with one of the reference sequences and few or no occurrences of nucleic acids with another of the reference sequences. FIG. 1C is a bar graph 130 that illustrates example relative abundance data. The horizontal axis 132 indicates the reference sequences Qₜ{t=1, T}. The vertical axis 134 indicates relative number of nucleic acids in the sample (designated by the symbol p) with each reference, with a higher value indicating a greater abundance of the associated reference sequence. Graph 130 indicates that Q₁ occurs in the sample 120 with a relative abundance ρ₁ indicated by bar 136a, and Q_{T} occurs in the sample 120 with a relative abundance ρ_{TT} indicated by bar 136b. The abundance distribution is represented by ρ=ρₜ, {t=1,T}.

A problem is that p is not measured directly during sequencing experiments, but must be inferred by a large number S of sequencing reads (simply called reads, herein), represented by the symbol qₛ {s=1, S}, where each sequence of each read is short compared to a reference sequence Q₁.

FIG. 1D is a block diagram that illustrates an example process to obtain reads from a sample and associate reads with reference sequences, according to an embodiment. The nucleic acids 122 in a sample are prepared for the sequencer in a wide variety of ways known in the art, often by de-naturing to release the nucleic acids, fragmentation to allow the short reads to begin sequencing from anywhere within the nucleic acid having the reference sequence, to hybridization or replication or amplification or size selection, among others, or some combination, which collectively are referenced herein as sample preparation process 140. The resulting short nucleic acids 150 are then sequenced with whatever bias or systematic variation are introduced by the sequencing process in sequencing machine 160. The reads qₛ {s=1, S} are recorded in a data structure 162 with a field holding data that represents each read sequence, such as field 162a for q₁ to field 162b for qₛ, among others indicated by ellipsis.

If each read were uniquely found in one and only one reference sequence, then one of the T reference sequences Q, can be associated with each read, as indicated by the data structure 180 which associates with each read qₛ {i∈1, S} an associated reference sequence Dₛ, with s∈{1, . . . , S} and where Dₛ=t with the {1, . . . , T}. Then a histogram of the distribution of the Dₛ among the T references sequences could be used as an approximation of the abundance distribution p, or corrected for the known or inferred non-random sampling introduced by processes 140 and machine 160—corrections represented by particular values for a parameters set designated θ. The adjusted abundances are designated A, and are based on the histogram counts for the associated reference sequences Dₛ and the corrections represented by values for θ.

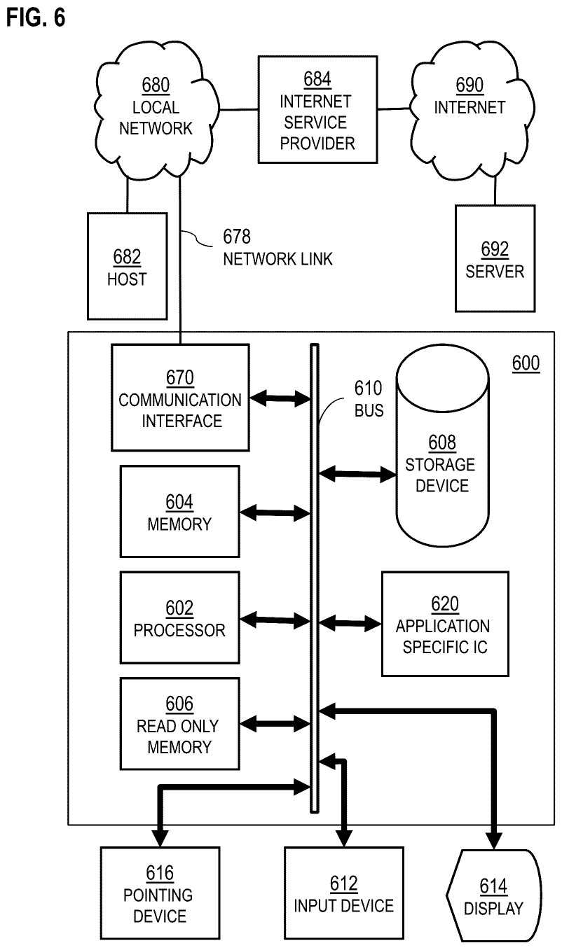

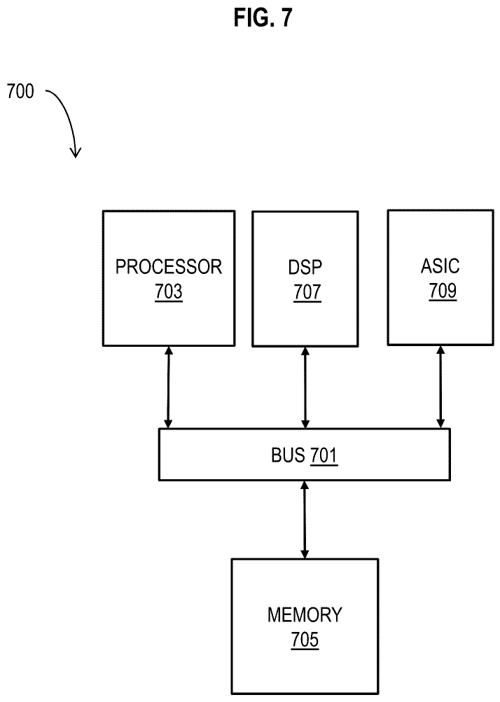

In FIG. 1D, the read sequences data structure 162 and associated reference sequences data structure 180 reside on a processing system 170, such as computer system described below with reference to FIG. 6 or one or more chip sets as described below with reference to FIG. 7, or some combination. The associated reference sequences Dₛ are converted to adjusted abundances A and used to infer a condition of a subject that contributed the sample 120 by condition inference module 172 implemented within the processing system 170. Although processes, equipment, and data structures are depicted in FIG. 1A through FIG. 1D as integral blocks in a particular arrangement for purposes of illustration, in other embodiments one or more processes or data structures, or portions thereof, are arranged in a different manner, on the same or different hosts, in one or more databases, or are omitted, or one or more different processes or data structures are included on the same or different hosts. For example, in various embodiments, one or both of the data structures 162, 180 or all or part of module 172, or some combination, reside within one or more chip sets in the sequencing machine 160.

Thus the clinical data comprises the adjusted counts A: {t=1, T) of the T reference sequences Q after correction for known systematic errors introduced by the processes 140 and machine 160. Based on the analysis of historical data or other training data, with either baseline (disease free) or known diseased conditions or known other conditions of interest, or some combination, the presence of a disease or other population differences is known to affect the count of at least one of the reference sequences, t=i but not, or much less, the counts of the other reference sequences t=k≠i. However, variation between runs or processing batches, which has nothing to do with disease state, can confound identification of disease by affecting the count Aᵢ.

Because the fetal or tumor fraction is so small, other confounding factors that affect the measured counts of the target are advantageously removed to form adjusted counts A; or Aⱼ. Several such adjustments are known in the art. As described here, additional adjustments are made to reduce the effects of noise, which is often about the same size as the condition of interest, especial for fetal or tumor or other small fraction signals.

In a first set of novel adjustments, smoothing is performed based on the observed variance of corrected abundances. This smoothing is done at a granularity of bins that each are made up of a reference sequence multiple kilobases long. The use of bins provides granularity finer than a chromosome for conditions such as localized mutations that are not associated with copy number of chromosomes or for cases in which the reads that indicate anomalous copy number are not found all along the chromosome. The number of sites (bases covered by a read alignment to the target reference sequence) in the kth non-overlapping bin of chromosome t is designated xₜₖ, which can also be considered the number of reads that fall in a bin. If reads are just a few hundred bases and bins are hundreds of kilobases, then it takes thousands of reads to completely cover a bin; and, one wants thousands of reads per bin to get confidence that one has a good sample that reflects the number of copies. The number of bins in chromosome t is nₜ. A number of sites normalized for comparison with data from other samples, such as using normalization described in a later section for an illustrated embodiment, are called a normalized number of sites and designated nxₜₖ. If this value is further corrected for various other effects, such as corrections described in a later section for an illustrated embodiment, the result is called a normalized bin score, designated nbₜₖ. During smoothing, the number of sites, or bin score, at each bin is combined with the number of sites, or bin score, in one or more adjacent bins.

According to some embodiments, an estimate of the spread of values in normalized number of sites (nxₜₖ) or bin scores (nbₜₖ), or both, in a training set, indicative of measurement system noise or reliability or consistency of a bin, is used in the smoothing. For example, in some embodiments, smoothing coefficients are set by functions that give more importance to bins with better performance (better performance is often seen with smaller spread).

A disadvantage of previous approaches is that users need to pre-select the region of interest; however, sometimes a copy number variation (CNV), or presence absence variation (PAV), or other structural aberration occurs in a location distal to the selected region or does not start and end exactly at the defined boundaries of the pre-selected region. Hidden Markov models (HMMs), when used in the correct manner, can address the issue. Hidden Markov models are not new to biological analysis and are commonly used to find genomic features; however, application of HMM methods according to the common practice does not lead to accurate identification of sub-chromosomal aneuploidy when the target aneuploidy is present at low fraction (such is the case in pre-natal and tumor screening from blood) because random noise looks like low fraction signal. The unique insight described here is that signal from the minority case (tumor/fetus) will mostly appear at a single fraction, say a, and that signal from the majority case (healthy-tissue/mother) will appear at fraction 1-a, but that random noise appears at many apparent-fraction levels. A new HMM topology has been designed to exploit this insight.

Therefore in some embodiments, a hidden Markov model (HMM) is constrained to attribute all non-normal rates of occurrence to a non-normal state at a fraction f that is complementary to a fraction (1-f) of the normal state, as described in more detail below. The observations are the adjusted (corrected or corrected and smoothed) abundances, e.g., Aₖₜ, of each base or bin or chromosome in the reference sequences based on the reads. While noise in the observations would be expected to be attributed to any of all possible states, an actual non-normal signal is expected to be attributed to only one of the states at the complementary fraction f. By introducing this constraint into the model hierarchy, the known methods for solving for the probabilities of hidden states of the model, such as the Viterbi process, can be used to detect the most probable non-normal state present in the sample. In the example embodiment, the states refer to a few normal and non-normal conditions at each of a discrete number of fractions. The discrete fractions are selected with a useful resolution, such as increments of a small percentage, e.g., selected in a range from about 0.5% to 5%, that span the expected fraction of the sample that exhibits the non-normal condition. According to the new topology, all states can transition to themselves and to a diploid state at the same fraction, say fraction f, but only the diploid state at one fraction, say fraction f, can communication with any other fraction and that is only with the diploid state at the complementary fraction (1−f).

In some embodiments both the smoothing based on spread in the training set and the hidden Markov model are used to compensate for noise.

### 2. METHOD

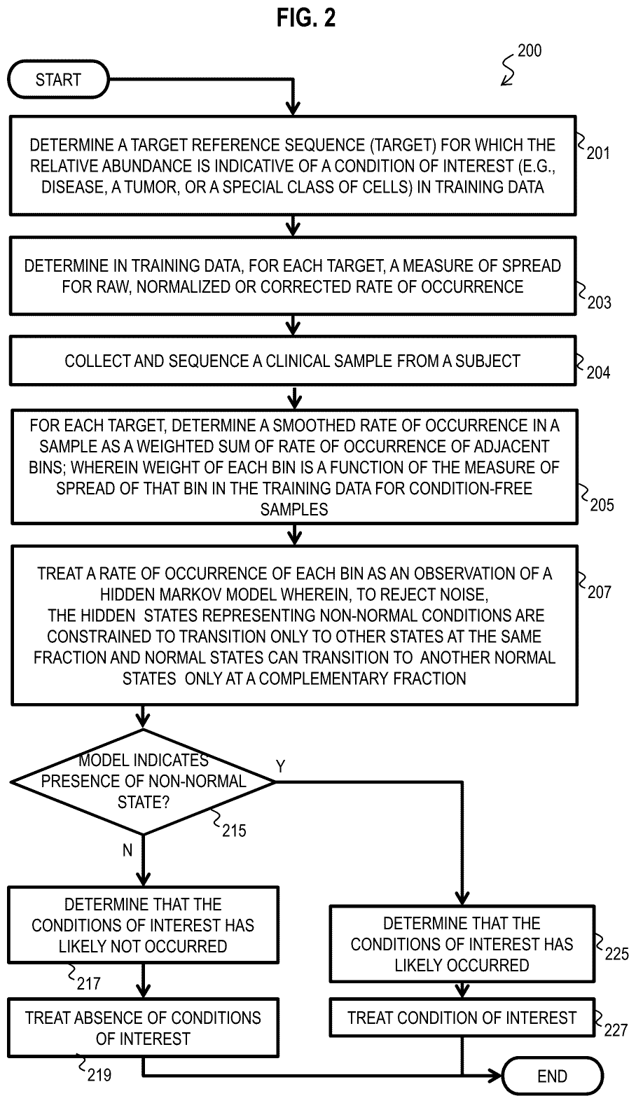

FIG. 2 is a flow chart that illustrates a method for 200 for forming and using noise compensation to increase the accuracy and sensitivity of DNA testing, according to an embodiment. Although steps are depicted in FIG. 2 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In step 201, a target reference sequence (called a target hereinafter for convenience) is determined among the reference sequences to be compared to the sequencing reads. Any method may be used to determine the target. In some embodiments the target is identified in scientific literature as a reference sequence for which the abundance is enhanced or diminished in a condition of interest (called simply a condition hereinafter for convenience), such as aneuploidy in which a whole chromosome is duplicated or absent, or tumors or diseased tissues that express one or more genes or proteins differently from normal cells.

In some embodiments, training data is used; and, the target is identified among the reference sequences by its correlation with the condition of interest known for the samples in the training data. Training data is a set of reference sequence abundances associated with the presence or absence of the condition of interest, among zero or more other conditions, also specified among multiple different subjects. In some embodiments, the training data is obtained by collecting biological samples from multiple subjects known to have the condition of interest or not, and sequencing the samples to determine a reference sequence abundance for all reference sequences for each subject. A reference sequence for which the abundances in the multiple samples are correlated with the occurrence of the condition of interest is then selected as a target reference sequence. As used herein, a subject is any higher order biological organism, including animals such as mammals such as humans.

Any biological sample from the subject that maintains the reference sequence abundances may be used for the training data, or for the clinical samples used in later steps described below. As used herein, biological samples include solid and fluid obtained from a subject. In various embodiments, biological samples may include tissue, organs, cells, protein or membrane extracts of cells, blood or biological fluids such as blood, serum, mucus, urine, ascites fluid or brain fluid (e.g., cerebrospinal fluid, csf).

In step 203, a measure of spread is determined for any of the raw or normalized or normalized and corrected abundances for any of the reference sequences. For example, in some embodiments, a variable trainingCountSpreadₜₖ is set equal to a value of a sample standard deviation of normalized number of counts, nxₜₖ, described above, over all samples in the training data. In some embodiment, a variable nbSpreadₜₖ is set equal to a value of a sample standard deviation of normalized and corrected bin score, nbₜₖ, described above, over all samples in the training data. Other embodiments use other estimates of spread, for example estimates of spread based upon median absolute deviation (see for example file Median_absolute_deviation in folder wiki in subdomain en of domain wikipedia in domain category org), inter-quartile range (see for example file Interquartile_range in folder wiki in subdomain en of domain wikipedia in domain category org), m-estimators (see for example file M-estimator in folder wiki in subdomain en of domain wikipedia in domain category org) or other alternative measures of variability.

In step 204 a clinical sample is collected from a subject and sequenced. Each clinical sample (called simply “sample” hereinafter for convenience) is to be evaluated for the occurrence of the condition of interest, and the following steps are repeated for each different clinical sample from the same subject or a different subject. A clinical sample is a biological sample taken from a subject for which it is not known whether the condition of interest occurs. Standard adjusted counts of the reference sequences for the sample are determined, e.g., retrieved from a local or remote data structure such as a database, or derived from measurements obtained directly using a sequencing machine 160, or some combination.

In step 205, a smoothed rate of occurrence of each target reference is determined as a weighted sum of one or more adjacent bins on the same chromosome. Local smoothing of data is nothing new and approaches like (un) weighted moving averages or medians (see for example file Moving_average in folder wiki in subdomain en of domain wikipedia in domain category org), splines, filters and many other methods are commonly used (see for example file Smoothing in folder wiki in subdomain en of domain wikipedia in domain category org). However, in step 205 the smoothing uses, at each bin, the performance of the bin over the training set of baseline (condition-free) samples. Thus in step 205, the weight of each bin is a function of the measure of spread of that bin in the training data. For example, a greater value of the weight is determined for a bin with a small spread, as that bin is expected to be more reliable either because it is less subject to noise or because that portion of the sequence has fewer variations among the normal training data, or some combination.

For example, smoothing of nxₜₖ is performed to produce a smoothed bin score, designated snxₜₖ, using Equation 1.1; and, smoothing of nbₜₖ is performed to produce a smoothed bin score, designated snbₜₖ, using Equation 1.2.

$$\\ \begin{matrix}{snx}_{tk} = \frac{{a}_{tk} {nx}_{tk} + {\sum }_{j = 1}^{W} [ {a}_{t ( k + j )} {nx}_{t ( k + j )} + {a}_{t ( k - j )} {nx}_{t ( k - j )} ]}{{w}_{tk}} & ( 1.1 )\end{matrix}$$

$$\\ \begin{matrix}{snb}_{tk} = \frac{{a}_{tk} {nb}_{tk} + {\sum }_{j = 1}^{W} [ {a}_{t ( k + j )} {nb}_{t ( k + j )} + {a}_{t ( k - j )} {nb}_{t ( k - j )} ]}{{w}_{tk}} & ( 1.2 )\end{matrix}$$

where wtₖ is given by Equation 1.3

$$\\ \begin{matrix}{w}_{tk} = {a}_{tk} + {\sum }_{j = 1}^{W} [ {a}_{t ( k + j )} + {a}_{t ( k - j )} ] & ( 1.3 )\end{matrix}$$

and W is a window size indicating how many bins on either side of bin k are included in the smoothing, aₜᵢ is the weights for that bin i on chromosome t, and (k+j) and (k−j) are not allowed past the ends of the chromosome.

In some embodiments, a bin is skipped if its performance is poor, e.g., its spread in the training data is greater than some threshold given by Threshold1 for training CountSpreadₜₖ and Threshold2 for nbSpreadₜₖ. This is accomplished by Equation 1.4

$$\\ \begin{matrix}{a}_{tk} = \{ \begin{matrix}0 & if trainingCountSpreadtk > Threshold 1 or nbSpreadtk > Threshold 2 \\ 1 & otherwise\end{matrix} & ( 1.4 )\end{matrix}$$

In some embodiments, the threshold is predetermined based on prior experience. In some embodiments, the threshold is based on the observed spreads in the training set, and is set below the largest spreads, e.g., at a value equal to one or two standard deviations of the spreads above the average spread, or at a certain percentile, e.g., at a value corresponding to the 75th percentile or 85th percentile of the spread values.

In some embodiments, the weight for the bin is the reciprocal of the spread, designated Spreadₜₖ, observed for that bin in the training data as given in Equation 1.5, where Spreadₜₖ=training CountSpreadₜₖ for smoothing nxₜₖ and Spreadₜₖ=nbSpreadₜₖ for smoothing nbₜₖ.

$$\\ \begin{matrix}{a}_{tk} = 1 / {Spread}_{tk} & ( 1.5 )\end{matrix}$$

In step 207, the adjusted bin occurrence rates are treated as observations in a special hidden Markov model with a constraint that helps to reduce the contribution of noise. The constraint is that all the non-normal fraction (f) reside in a non-normal condition at a complementary fraction to the fraction (1−f) of the normal condition. This has the effect of requiring all the non-normal fraction to occur at a single condition. This has not been done before in the analysis of sequence data for small fractions in a sample.

In probability theory, a Markov model is a stochastic model used to model randomly changing systems where it is assumed that future states depend only on the present state and not on the sequence of events that preceded it (that is, it assumes the Markov property). Generally, this assumption enables reasoning and computation with the model that would otherwise be intractable. In simpler Markov models (like a Markov chain), the state is directly visible to the observer, and therefore the state transition probabilities are the only parameters. In a hidden Markov model (HMM), the state is not directly visible, but output, dependent on the state, is visible. Each state has a probability distribution over the possible outputs. That is, for a given state there is an asserted probability for each of several possible outputs. Therefore the sequence of outputs gives some information about the sequence of states of a HMM. Note that the adjective ‘hidden’ refers to the state sequence through which the model passes, not to the parameters of the model, which are the probabilities of transitioning between states, the probabilities of a particular output given a current state, and any relationships among the states. The model is still referred to as a ‘hidden’ Markov model even if these parameters are known exactly.

As applied here to the compensation for noisy data, an HMM is constructed as follows. The hidden states represent the unknown fractions of the sample that represents each of two or more conditions (e.g., diploid, which is normal, and trisomy). A plurality of states are defined for each condition in each of a discreet number of different fractions. For example, states are defined for the subject's normal cells making up 85%, 90%, 95%, and 100% of the sample; and states are defined for the tumor or fetal cells making up 15%, 10%, 5% and 0% of the sample.

The HMM defined here has several novel properties. As stated above, all states can transition to themselves and to a diploid state at the same fraction, say fraction f, but only the diploid state at one fraction, say fraction f, can communication with any other fraction and that is only with the diploid state at the complementary fraction (1−f). Given a condition at a given fraction of a sample (i.e., given one state) the probability that the next read is from the same state can be given as f and the probability that the next read is from the normal state is 1−f. By restricting communication between states representing different fractions, the HMM is forced allocate non-normal states (e.g., non-diploid for most chromosomes or non-monosomy in special cases like the Human sex chromosomes) to a single level of real signal while random noise remains at the diploid state. Second, by selectively allowing communication between normal states such that normal states a and b are allowed to communicate if fraction_a+fraction_b=1, the majority source (for example, the maternal DNA in pre-natal screening) and the minority source (for example, the fetal DNA in pre-natal screening) are both allowed to show copy number. Third, a minority fraction (hence fetal fraction in some embodiments) estimate is a product of standard methods to decode HMM (to assign fraction and copy number states to each observed data point).

The parameters are determined using standard HMM methods. For example, the model can be parameterized using machine learning techniques like Baum Welch HMM Training (see for example file Baum % E2%80%93Welch_algorithm in folder wiki in subdomain en of domain wikipedia in category org, at the time of this writing, the entire contents of which are hereby incorporated by reference as if fully set forth herein) or Viterbi HMM Training, as described in the file cited above. Alternatively, the HMM can be parameterized using knowledge of the biological characteristics of the disease, syndrome or trait under investigation. For example, if it is known that DiGeoge, Angleman, Wolf-Hirchhorn, and CriDuChat Syndromes in Human Infants involve amplification of at least two megabases of DNA, duration modeling can be used to set the return probability of each non-diploid state-if bins of 100 kilobases are being used then the probability of, say, remaining in a trisomy state could be set to 1−(1/2,000,000). Another example of using biological knowledge is the incidence of the phenomena under study can motivate the choice of initial probability and the target sequence.

The probability that the observations are due to a state representing condition C at fraction f is represented by the expectation density e, a function of both, designated e(C,f). Given the parameters of the HMM, e(C,f) can then be determined based on the distribution of nucleotides or bins or chromosomes obtained, using any of several HMM methods known in the art. Examples of decoding algorithms include Viterbi (see for example file Viterbi algorithm in folder wiki in subdomain en of domain wikipedia in category org, at the time of this writing, the entire contents of which are hereby incorporated by reference as if fully set forth herein) and forward-backward (see for example file Forward%E2%80%93backward_algorithm in folder wiki in subdomain en of domain wikipedia in category org, at the time of this writing, the entire contents of which are hereby incorporated by reference as if fully set forth herein). Given the current state is a particular conditions, e.g., a trisomy at 5% fraction, the probability of reading each base, or bin or chromosome can be computed for an output probability distribution. These probability distributions are the parameters of the model. The solution of the HMM provides a value for e(C,f) for all the hidden states.

In step 215, it is determined whether the HMM solution indicates the presence of a non-normal state. In some embodiments, this determination is based on the most probable non-normal state, e.g., the condition Cmax at fraction fmax, for which e(Cmax,fmax)>e(C,f) for any other non-normal state (C,f). If fmax is zero, then it is determined that the condition is not present. In some embodiments, the values of e(C,f) are summed for all small fractions for each conditions, e.g., the maximum is selected among the non-normal conditions for the sum over a set of f values f=f1, f2, f3 . . . for two or more fractions less than half and not equal to zero for one condition.

If it is determined in step 215 that the presence of a non-normal state is not indicated, then in step 217 it is determined that the conditions of interest likely has not occurred in the subject. In step 219 the subject is treated as if the condition has not occurred. For example, the information is presented on a display and conveyed to the subject.

If, however, it is determined in step 215 that the presence of a non-normal state is indicated, then in step 226 it is determined that the conditions of interest likely has indeed occurred in the subject. In step 227, the condition of interest is treated by any method known for the condition of interest. For example, the information is presented on a display and conveyed to the subject, and a treatment plan is presented, or some combination.

Using the method 200, a small amount of cells indicative of the condition of interest can be detected in a sample with many cells not in such a condition. Both the sensitivity and accuracy are improved, as will be demonstrated in the following particular embodiments.

### 3. EXAMPLE EMBODIMENTS

Schnert et al 2011 and Biananchi et al 2014 describe methods to identify aneuploidy from maternal blood samples, thus avoiding an expensive and dangerous invasive procedure; however, they use a normalization functions with weights selected from the values 0 and 1 instead of over the one dimensional real numbers, [symbol] . In this example, each reference sequences is most or all of an entire chromosome, the condition is Down's syndrome which is indicated by aneuploidy in human chromosome 21 (T21); and thus chromosome 21 is the target. The prior art derivation is repeated here with notation changed to avoid ambiguity with the notation used above. In some embodiments, aneuploidy of a different chromosome, such as 5, 13, 18 or 21, is the condition of interest.

The read sequences q are processed as follows. Reads are aligned to the masked or unmasked Human genome assembly (version hg.19) assembly, which is the entire genome sequence, not just the target region. A read is aligned with a reference sequence at standard criteria (an example would be even with two mismatches provided there are no gaps, and with a read being dropped if it does not align according to this definition, or if it aligns with more than one location on the reference sequence). The number of sites (bases covered by a read alignment to the one reference sequence) in the kth non-overlapping bin of size 100 kilobases (kb, 1 kb=10³ nucleotides) of chromosome t is designated xₜₖ. The number of bins in chromosome t is nₜ. The percentage of G or C nucleotides in the sequence covered by the kth bin of chromosome t is designated GCₜₖ. In the example embodiment, the target is a chromosome or a region of a chromosome and the covariates are the chromosomes in a set V of robust chromosomes that includes all chromosomes except common targets 13, 18, 21, x and y, i.e., V={all chromosomes}\{13, 18, 21, x, y}.

Because the fetal fraction is so small, other confounding factors that affect the measured counts of the target are advantageously removed, as described here, to form adjusted counts Aᵢ or Aⱼ.

Certain chromosome bins are excluded from further analysis due to observed high variability among baseline (disease free) samples, such that the variability contains little information about aneuploidy state. This was done by manual inspection. Excluded Regions of the Human genome (version hg.19 unmasked) were chromosome y: bases 0-2,000,000; bases 10,000,000-13,000,000; and bases 23,000,000-end of chromosome y.

In addition, corrections are applied for differences in the total number of sequences generated, according to Equation 2.1.

$$\\ \begin{matrix}{nx}_{tk} = \frac{{x}_{tk}}{{\sum }_{j ∈ U} {\sum }_{k = 1}^{{n}_{j}} {x}_{tk}} & ( 2.1 )\end{matrix}$$

Corrections are applied for bin effects according to Equation 2.2.

$$\\ \begin{matrix}{b}_{tk} = \frac{{nx}_{tk}}{α {GWP}_{tk} + β ′} & ( 2.2 )\end{matrix}$$

where GWPₜₖ is the median of {nxₜₖ} over all samples in the training data. In the example embodiment, the training samples included sex chromosomes with female fetuses; but, in other embodiments, other samples are included in the training data. The denominator provides an estimate of the expected count in the bin based on a linear model for expected value of nxₜₖ, in which the coefficients α and β′ (two of the parameters generally referenced above as θ) are determined using a robust Huber-M estimate as implemented in the rim( ) function available from MASS R library at World Wide Web domain r-project in the super domain org hosted at the time of this writing by the Vienna University of Economics and Business, Vienna, Austria.

In Illumina sequencing, coverage is heavily biased by GC content and the resulting bias dominates the small fetal signals of interest. To compensate, a sample-specific GC bias curve is generated in which bₜₖ=gcBias(GCₜₖ) using loess regression on bins from the covariate set V of baseline or disease free training samples, and applied according to Equation 2.3.

$$\\ \begin{matrix}{nb}_{tk} = {b}_{tk} - Median \{ gcBias ( {GC}_{lm} ) ∀ l , m such that ⌊ \frac{{GC}_{tk}}{2} ⌋ = ⌊ \frac{{GC}_{lm}}{2} ⌋ , l ∈ V \} & ( 2.3 )\end{matrix}$$

where nbₜₖ is the bin score (normalized and corrected bin count), └a┘ indicates the floor function, which produces the largest integer not greater than the enclosed quantity α. The parameters of gcBias are included among the general parameters θ, for this embodiment.

Confounding maternal sub-chromosomal amplifications and deletions are removed by excluding bins with large deviations from a chromosomal median. To express this correction, the following notation is introduced.

$$\\ {c}_{t} = Median ( \{ {nb}_{tk} \} )$$

$$\\ {ad}_{tk} = \vert {nb}_{tk} - {c}_{t} \vert$$

$$\\ {MAD}_{t} = 1.4826 * Median ( {ad}_{tk} )$$

Where MAD, is the standard deviation of the adₜₖ when the nbₜₖ are normally distributed. The final adjusted counts A, with this correction are then given by Equation 2.4.

$$\\ \begin{matrix}{A}_{t} = Mean ( {nb}_{tm} ) over all m such that \vert {nb}_{tm} - {c}_{t} \vert < \sim {MAD}_{t} & ( 2.4 )\end{matrix}$$

As stated above, Sehnert et al. (2011) and Biananchi et al. (2014) normalize with weights that are selected from the integers 0 and 1, as described here. The final normalization consists of dividing A; by an internal control consisting of a linear combination of the Aⱼ from robust set U=V†i. Coefficients of the linear combination are set during training and remain constant for all clinical samples. The normalizing function in this case is a denominator Di that parallels Yᵢ described above with reference to Equation 1a.

$$\\ \begin{matrix}{D}_{i} = {\sum }_{j ∈ V {}_{} \backslash {}_{} i} {}_{} {δ}_{j} {A}_{j} , {δ}_{j} ∈ \{ 0 , 1 \} & ( 2.4 \text{.1} a )\end{matrix}$$

$$\\ \begin{matrix}{δ}_{j} = arg min \{ SampleVariance of \frac{{A}_{i}}{{\sum }_{j ∈ V {}_{} \backslash {}_{} i} {}_{} {δ}_{j} {N}_{j}} in training data \} , & ( 2.4 \text{.1} b )\end{matrix}$$

$$\\ j ∈ V {}_{} \backslash {}_{} i$$

$$\\ \begin{matrix}Normalized adjusted target count is {B}_{i} = \frac{{A}_{i}}{{D}_{i}} & ( 2.4 \text{.1} c )\end{matrix}$$

The δⱼ that solve Equation 2.4.1b are determined by brute force, as mentioned above, and takes days to weeks; but, can be reduced to hours if the set of all possible combinations is reduced by, say, only allowing four of the coefficients to be non-zero. However, this time savings might lead to a decrease in accuracy of the solution. In some embodiments, it was realized that it was tractable, faster and more accurate to allow the δⱼ to take any real value, positive or negative. In an example embodiment, δⱼ is allowed to be member of the one dimensional real number set, [symbol] (δⱼ∈ [symbol] ). Those weights δⱼ are derived to minimize the variance of the Di using standard techniques for optimization.

In step 203, in a training set, the above processing is done and the spread is determined for the values of nxₜₖ or nbₜₖ or Aₜₖ, or some combination, over the training set. The spread is then used in step 205 to smooth the observations of bin counts or bin scores on a sample collected in step 204.

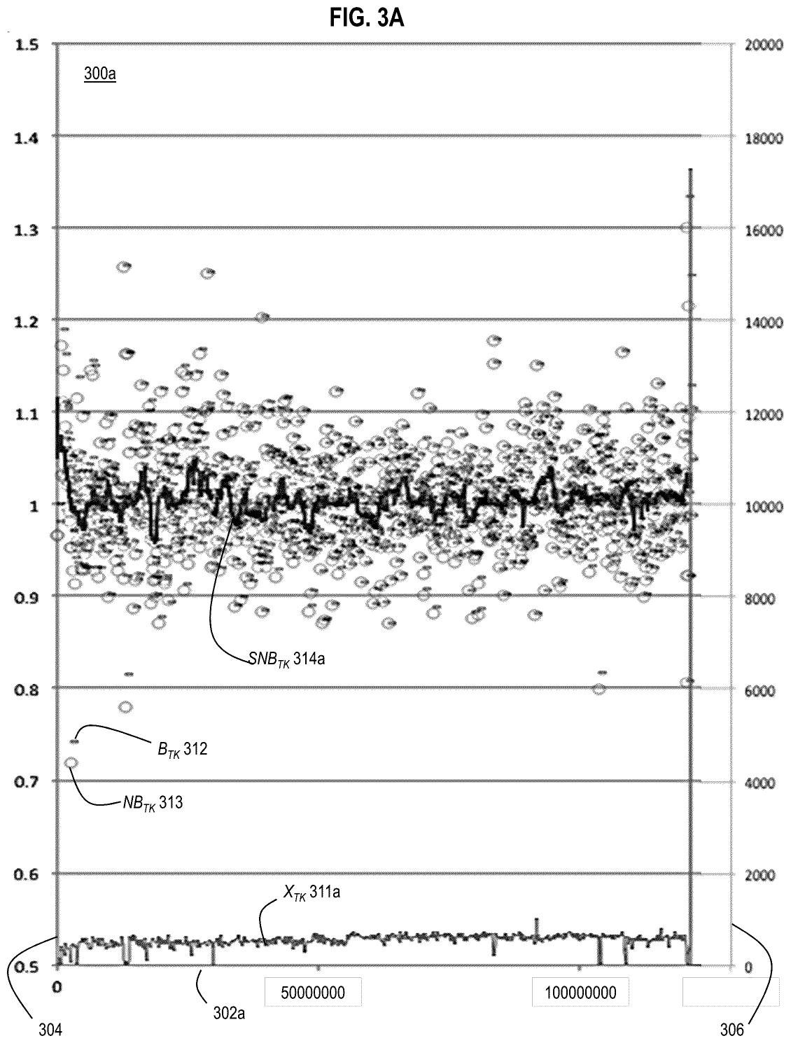

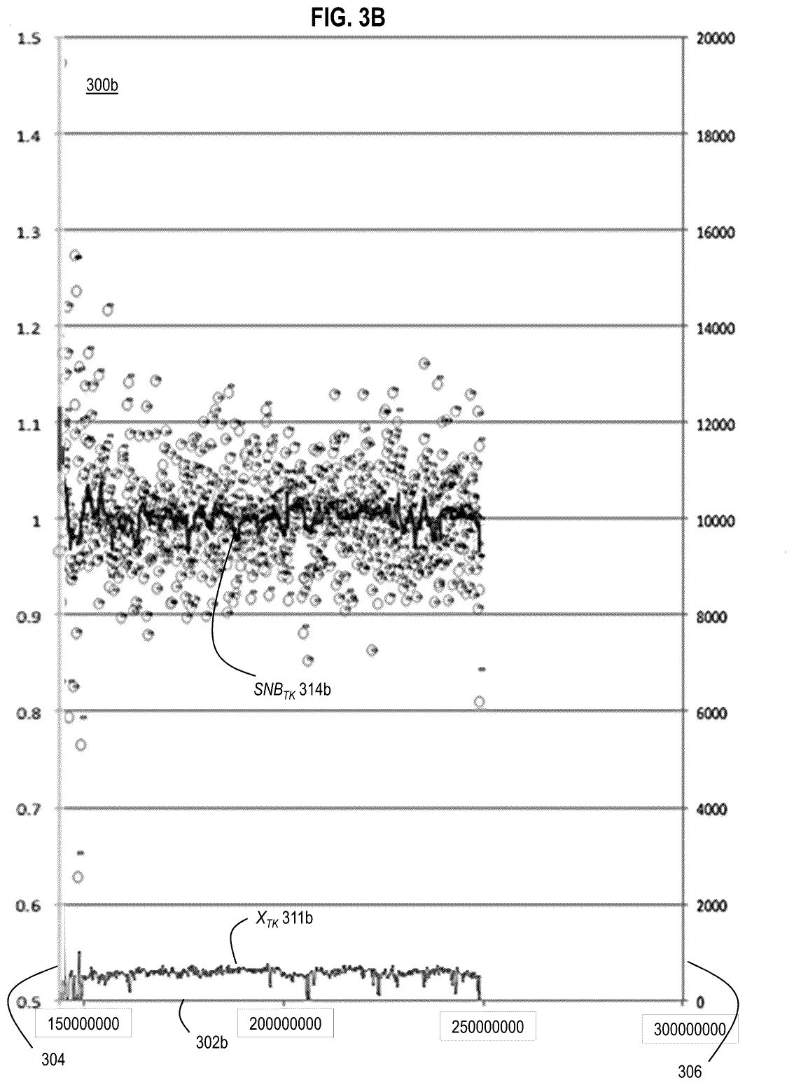

FIG. 3A and FIG. 3B are graphs of successive portions of a plot that illustrates example distribution of abundances of regions of a chromosome among millions of reads during processing, according to an embodiment. The right vertical axis 306 indicates abundance in number of bases covered by a read alignment to the target reference sequence (e.g., values for xₜₖ). The left vertical axis is the relative abundance after normalizing (e.g., by the average or median number of reads per bin) so that the observations cluster around 1. The horizontal axis 302a and 302b indicates the position of the bin in the reference sequence in units of number of bases on chromosome Chr5. Note that FIG. 3A with horizontal axis 302a represents a left portion of a plot and FIG. 3B with horizontal axis 302b represents a non-overlapping right portion of the same plot. The values of xₜₖ relative to the right vertical axis 306 is given by trace 311a in FIGS. 3A and 311b in FIG. 3B. The normalized and bin corrected values bₜₖ are represented by the dashes 312 relative to the left vertical axis 304 in both FIG. 3A and FIG. 3B. The further corrected values of nbₜₖ are represented by the dashes 312 relative to the left vertical axis 304 in both FIG. 3A and FIG. 3B. After smoothing in step 203 using the spread of nbₜₖ from the training set and equation 1.5, the values of snbₜₖ relative to the left vertical axis 304 is given by trace 314a in FIGS. 3A and 314b in FIG. 3B. The different traces show the incremental reduction of noise as each step is applied to bin data. The original count data (xₜk in traces 311a, 311b) had a standard deviation over 166; but, the standard deviation is reduced to 0.0372 by the previous methods to produce nbₜₖ plotted as points 313. The new method described here results in snbₜₖ traces 314a, 314b and variability is reduced to one third of the previous value (standard deviation=0.0113). The noise is evidently substantially reduced without eliminating variations in abundance among the portions of the reference sequence. This is appropriate with copy number variations that involve many bins.

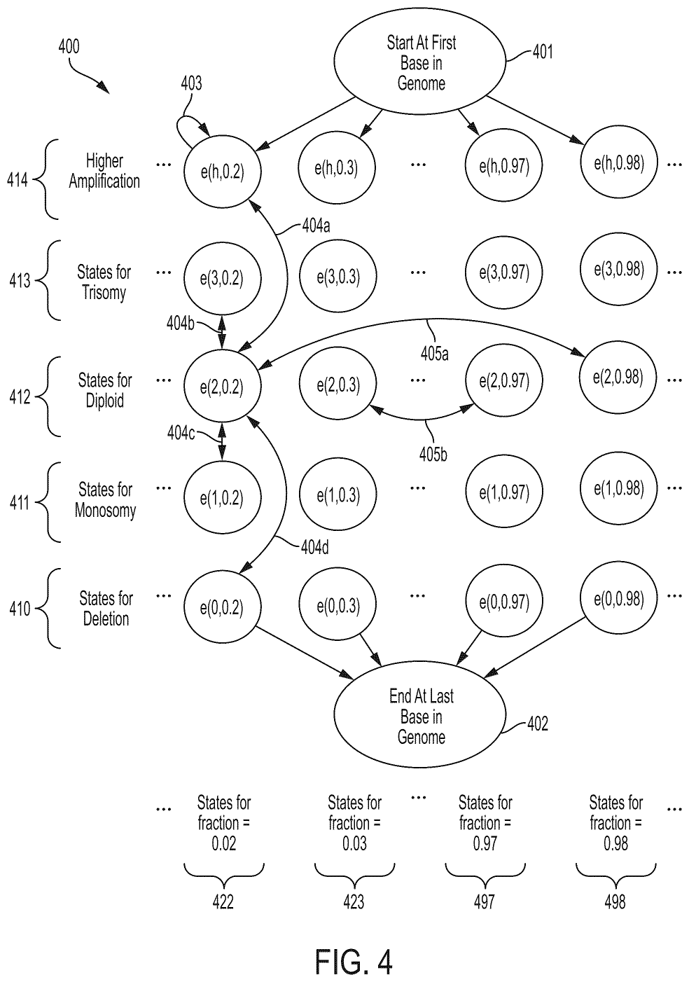

In step 207, whether or not smoothing is performed in step 205, the data is used as observations derived from a hidden Markov Model with special characteristics, as described above. FIG. 4 is a block diagram that illustrates features of a new hidden Markov model (HMM) 400 that compensates for noise in samples with contribution from a small fraction, according to an embodiment. The model is made up of multiple states that each represent a particular combination of one of five conditions and one of multiple different sample fractions. The different conditions are arranged in different rows of a two dimensional (2D) graphic representation and the different fractions are arranged in different columns. In the illustrated example, the conditions of interest are the relative number of copies for each chromosome or portion thereof (e.g., bin or individual base). Therefore the conditions represented by different rows are deletions (missing portions) in row 410, monosomy (one copy of each portion) in row 411, diploid (two copies of each portion) in row 412, trisomy (three copies of each portion) in row 413, and higher amplification (4 or more copies of each portion) in row 414. The different sample fraction exhibiting each condition represented by different columns have a granularity of 1%. In some embodiments, every possible sample fraction is represented in the model and there are 101 different columns. However, in some embodiments, it is known that the minority fraction (<50%) of the fetus or tumor or other source in the sample is below some maximum minority fraction, and minority fractions greater than that maximum minority fraction are not included in the model. However, for each minority fraction f, there is a majority fraction 1-f also included as a column of states in the HMM. Thus the model includes states in pairs of complementary fractions. Included in the example model are minority fraction 2% (0.02) in column 422 and fraction 3% (0.03) in column 423 and their complementary majority fractions 98% (0.98) in column 498 and 98% (0.97) in column 497, respectively, among others indicated by ellipses. For convenience each state will be designated by its row, column pair in parenthesis.

The parameters of the model include the probability that, given one observation (read) is due to a particular state, the next read is due to a different state. The probabilities of changing states per read are represented in FIG. 4 by the arrows. Not all state change probabilities are shown, in order to avoid obscuring the diagram. The probability that the next observation is from the same state is given by an arrow that begins and ends on the same state, as illustrated for example by arrow 403 for state (414,422) i.e., on row 414 column 422. Each state has such a probability of persistence. That probability is expected to be the product of sample fraction of the state multiplied by the number of copies represented by the state (normalized so the total probability is 1). According to the novel topology, the only other transitions allowed for the non-normal conditions are transitions with the normal condition (e.g., diploid in the illustrated example) on the same fraction. Such transitions are shown for column 422 only, as double headed arrows connecting to state (412, 422) including arrows 404a with state (414, 422), arrows 404b with state (413, 422), arrows 404c with state (411, 422), and arrows 404d with state (410, 422). The normal state at the current fraction can also transition with the normal state at the complementary fraction. Such transitions are shown for column 422 and 423 only. Arrow 405a represents the transition between the normal state at fraction 2% (412, 422) and the complementary normal state at fraction 98% (412, 498). Similarly, arrow 405b represents the transition between the normal state at fraction 3% (412, 423) and the complementary normal state at fraction 97% (412, 497). By fitting this HMM to the observations using the standard techniques, a value is obtained for each state which indicates the probability that the sample is in that state.

Item 401 represents a previous state of the model and item 402 represents a subsequent state of the model during an iterative solution process. The resolution is at the bin level.

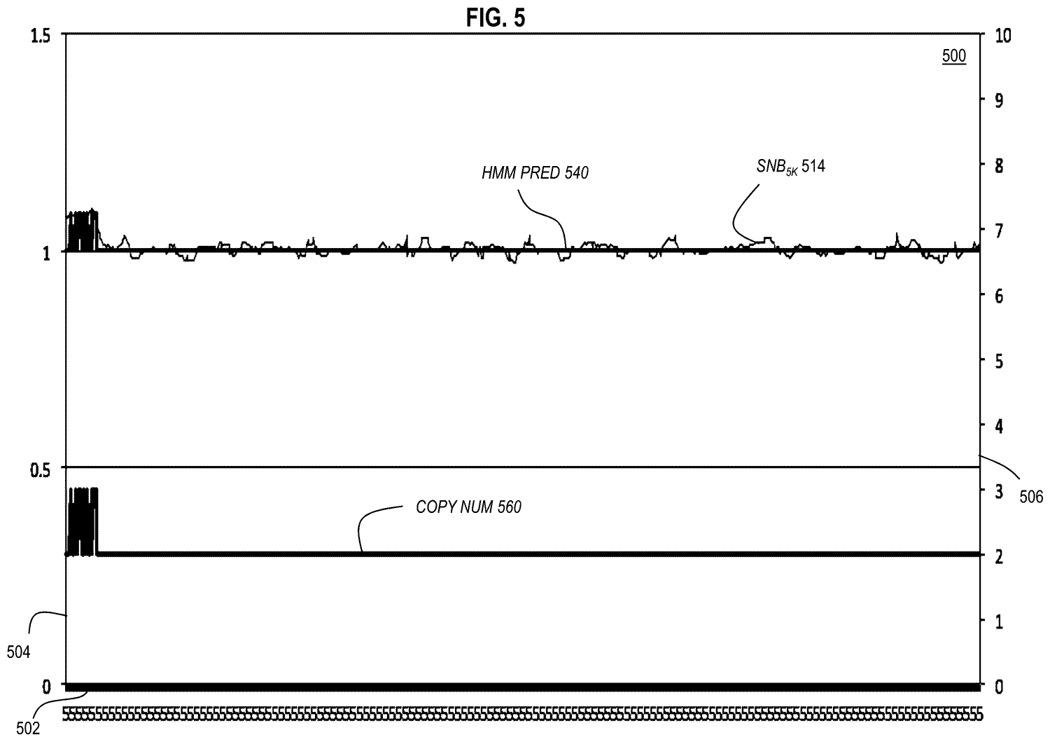

In step 215 it is determined whether the model indicates the presence of a non-normal state. FIG. 5 is a graph 500 that illustrates model solution and corresponding copy number for portions of a chromosome, according to an embodiment. The horizontal axis is position along Chromosome 5 in number of bins. The left vertical axis 502 indicates the relative abundance after normalizing (e.g., by the average number of reads per bin) so that the observations cluster around 1. The right vertical axis 506 indicates copy number. Trace 514 is relative abundance of the observed smoothed normalized and corrected bin data for chromosome 5 (snb₅ₖ). This trace is elevated at the left side of the plot near the beginning of Chromosome 5. The solution to the HMM model depicted in FIG. 4 is that the most probable state is a trisomy state for many of the bins at the beginning of Chromosome 5, as indicated by trace 560 plotted relative to the right axis 506. The model says the observations are consistent with 3 copies of many of these first portions of the Chromosome and 2 copies of the remainder. This solution predicts the smoothed corrected and normalized abundance given by HMM predicted abundance trace 540, plotted relative to left axis 504. As can be seen, the prediction closely follows the smoothed corrected and normalized abundance given by trace 514.

FIG. 5 shows the method described here identifying a possible amplification at the start of Human chromosome 5, which is associated with Cri-Du-Chat Syndrome. The trace 540 is an optimal decoding of the data by the HMM which signals duplication at the beginning of the chromosome.

The use of this special HMM exploits the featured recognized herein—that in a sample, noise occurs at many different low levels and may commonly appear as the same magnitude as a fetal or tumor signal at fraction between 0.1-10%; while, in contrast, signal from a tumor or fetal DNA appears tightly around a constant fraction.

### 4. HARDWARE OVERVIEW

FIG. 6 is a block diagram that illustrates a computer system 600 upon which an embodiment of the invention may be implemented. Computer system 600 includes a communication mechanism such as a bus 610 for passing information between other internal and external components of the computer system 600. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit)). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 600, or a portion thereof, constitutes a means for performing one or more steps of one or more methods described herein.

A sequence of binary digits constitutes digital data that is used to represent a number or code for a character. A bus 610 includes many parallel conductors of information so that information is transferred quickly among devices coupled to the bus 610. One or more processors 602 for processing information are coupled with the bus 610. A processor 602 performs a set of operations on information. The set of operations include bringing information in from the bus 610 and placing information on the bus 610. The set of operations also typically include comparing two or more units of information, shifting positions of units of information, and combining two or more units of information, such as by addition or multiplication. A sequence of operations to be executed by the processor 602 constitute computer instructions.

Computer system 600 also includes a memory 604 coupled to bus 610. The memory 604, such as a random access memory (RAM) or other dynamic storage device, stores information including computer instructions. Dynamic memory allows information stored therein to be changed by the computer system 600. RAM allows a unit of information stored at a location called a memory address to be stored and retrieved independently of information at neighboring addresses. The memory 604 is also used by the processor 602 to store temporary values during execution of computer instructions. The computer system 600 also includes a read only memory (ROM) 606 or other static storage device coupled to the bus 610 for storing static information, including instructions, that is not changed by the computer system 600. Also coupled to bus 610 is a non-volatile (persistent) storage device 608, such as a magnetic disk or optical disk, for storing information, including instructions, that persists even when the computer system 600 is turned off or otherwise loses power.

Information, including instructions, is provided to the bus 610 for use by the processor from an external input device 612, such as a keyboard containing alphanumeric keys operated by a human user, or a sensor. A sensor detects conditions in its vicinity and transforms those detections into signals compatible with the signals used to represent information in computer system 600. Other external devices coupled to bus 610, used primarily for interacting with humans, include a display device 614, such as a cathode ray tube (CRT) or a liquid crystal display (LCD), for presenting images, and a pointing device 616, such as a mouse or a trackball or cursor direction keys, for controlling a position of a small cursor image presented on the display 614 and issuing commands associated with graphical elements presented on the display 614.

In the illustrated embodiment, special purpose hardware, such as an application specific integrated circuit (IC) 620, is coupled to bus 610. The special purpose hardware is configured to perform operations not performed by processor 602 quickly enough for special purposes. Examples of application specific ICs include graphics accelerator cards for generating images for display 614, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 600 also includes one or more instances of a communications interface 670 coupled to bus 610. Communication interface 670 provides a two-way communication coupling to a variety of external devices that operate with their own processors, such as printers, scanners and external disks. In general the coupling is with a network link 678 that is connected to a local network 680 to which a variety of external devices with their own processors are connected. For example, communication interface 670 may be a parallel port or a serial port or a universal serial bus (USB) port on a personal computer. In some embodiments, communications interface 670 is an integrated services digital network (ISDN) card or a digital subscriber line (DSL) card or a telephone modem that provides an information communication connection to a corresponding type of telephone line. In some embodiments, a communication interface 670 is a cable modem that converts signals on bus 610 into signals for a communication connection over a coaxial cable or into optical signals for a communication connection over a fiber optic cable. As another example, communications interface 670 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 670 sends and receives electrical, acoustic or electromagnetic signals, including infrared and optical signals, that carry information streams, such as digital data.

The term computer-readable medium is used herein to refer to any medium that participates in providing information to processor 602, including instructions for execution. Such a medium may take many forms, including, but not limited to, non-volatile media, volatile media and transmission media. Non-volatile media include, for example, optical or magnetic disks, such as storage device 608. Volatile media include, for example, dynamic memory 604. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 602, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 602, except for carrier waves and other signals.

Logic encoded in one or more tangible media includes one or both of processor instructions on a computer-readable storage media and special purpose hardware, such as ASIC 620.

Network link 678 typically provides information communication through one or more networks to other devices that use or process the information. For example, network link 678 may provide a connection through local network 680 to a host computer 682 or to equipment 684 operated by an Internet Service Provider (ISP). ISP equipment 684 in turn provides data communication services through the public, world-wide packet-switching communication network of networks now commonly referred to as the Internet 690. A computer called a server 692 connected to the Internet provides a service in response to information received over the Internet. For example, server 692 provides information representing video data for presentation at display 614.

The invention is related to the use of computer system 600 for implementing the techniques described herein. According to one embodiment of the invention, those techniques are performed by computer system 600 in response to processor 602 executing one or more sequences of one or more instructions contained in memory 604. Such instructions, also called software and program code, may be read into memory 604 from another computer-readable medium such as storage device 608. Execution of the sequences of instructions contained in memory 604 causes processor 602 to perform the method steps described herein. In alternative embodiments, hardware, such as application specific integrated circuit 620, may be used in place of or in combination with software to implement the invention. Thus, embodiments of the invention are not limited to any specific combination of hardware and software.

The signals transmitted over network link 678 and other networks through communications interface 670, carry information to and from computer system 600. Computer system 600 can send and receive information, including program code, through the networks 680, 690 among others, through network link 678 and communications interface 670. In an example using the Internet 690, a server 692 transmits program code for a particular application, requested by a message sent from computer 600, through Internet 690, ISP equipment 684, local network 680 and communications interface 670. The received code may be executed by processor 602 as it is received, or may be stored in storage device 608 or other non-volatile storage for later execution, or both. In this manner, computer system 600 may obtain application program code in the form of a signal on a carrier wave.

Various forms of computer readable media may be involved in carrying one or more sequence of instructions or data or both to processor 602 for execution. For example, instructions and data may initially be carried on a magnetic disk of a remote computer such as host 682. The remote computer loads the instructions and data into its dynamic memory and sends the instructions and data over a telephone line using a modem. A modem local to the computer system 600 receives the instructions and data on a telephone line and uses an infra-red transmitter to convert the instructions and data to a signal on an infra-red a carrier wave serving as the network link 678. An infrared detector serving as communications interface 670 receives the instructions and data carried in the infrared signal and places information representing the instructions and data onto bus 610. Bus 610 carries the information to memory 604 from which processor 602 retrieves and executes the instructions using some of the data sent with the instructions. The instructions and data received in memory 604 may optionally be stored on storage device 608, either before or after execution by the processor 602.

FIG. 7 illustrates a chip set 700 upon which an embodiment of the invention may be implemented. Chip set 700 is programmed to perform one or more steps of a method described herein and includes, for instance, the processor and memory components described with respect to FIG. 6 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 700, or a portion thereof, constitutes a means for performing one or more steps of a method described herein.

In one embodiment, the chip set 700 includes a communication mechanism such as a bus 701 for passing information among the components of the chip set 700. A processor 703 has connectivity to the bus 701 to execute instructions and process information stored in, for example, a memory 705. The processor 703 may include one or more processing cores with each core configured to perform independently. A multi-core processor enables multiprocessing within a single physical package. Examples of a multi-core processor include two, four, eight, or greater numbers of processing cores. Alternatively or in addition, the processor 703 may include one or more microprocessors configured in tandem via the bus 701 to enable independent execution of instructions, pipelining, and multithreading. The processor 703 may also be accompanied with one or more specialized components to perform certain processing functions and tasks such as one or more digital signal processors (DSP) 707, or one or more application-specific integrated circuits (ASIC) 709. A DSP 707 typically is configured to process real-world signals (e.g., sound) in real time independently of the processor 703. Similarly, an ASIC 709 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 703 and accompanying components have connectivity to the memory 705 via the bus 701. The memory 705 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 705 also stores the data associated with or generated by the execution of one or more steps of the methods described herein.

### 5. ALTERNATIONS, EXTENSIONS AND MODIFICATIONS

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

### 6. REFERENCES

Sehnert et al. 2011 Clinical Chemistry 57 (7): 1042-1049.
Biananchi et al. 2014 N Engl J Med 370 (9): 799-808.

What is claimed is:

1. A method comprising:
collecting a clinical biological sample with a minority component and a majority component, wherein the majority component has a known sequence in a target bin;
measuring with a nucleotide sequencer amount data that indicates an amount in the clinical biological sample of reads in the target bin;
selecting a candidate fraction f for the minority component from a plurality of pre-selected fractions less than 0.5;
determining automatically on a processor a condition of the minority component based on an amount of the minority component in the target bin using a Hidden Markov Model for a hidden state that indicates a read is from the minority component or the majority component, wherein the HMM is constrained to only allow a change of state if the read indicates the known sequence in the target bin, and the probability to change to the minority fraction is f and the probability to change to the majority component is 1-f; and
presenting on a display, output data that indicates a condition for the minority component.

2. The method as recited in claim 1, further comprising
selecting a different candidate fraction f′ and
determining the condition of the minority component for a different candidate fraction f′,
wherein said step of presenting the output data further comprises presenting output data that indicates the condition for the minority component based on the selected candidate fraction f or f′ for which the HMM better fits the amount data.

3. The method as recited in claim 1, wherein the amount data for the target bin is a weighted sum of amounts in the target bin and bins on either side of the target bin.

4. The method as recited in claim 3, wherein weights for the weighted sum are based on spreads of amounts in the target bin and of amounts in the bins on either side of the target bin among training data collected from a plurality of biological samples having only the majority component.

5. A system comprising:
a nucleic acid sequencing device;
at least one processor; and
at least one memory including one or more sequences of instructions,
the at least one memory and the one or more sequences of instructions configured to, with the at least one processor, cause an apparatus to perform at least the following,
obtaining, from the sequencing device, amount data that indicates an amount in a clinical biological sample of reads in the target bin, wherein the biological sample has a minority component and a majority component, wherein the majority component has a known sequence in a target bin;
selecting a candidate fraction f for the minority component from a plurality of pre-selected fractions less than 0.5;
determining automatically on a processor a condition of the minority component based on an amount of the minority component in the target bin using a Hidden Markov Model for a hidden state that indicates a read is from the minority component or the majority component, wherein the HMM is constrained to only allow a change of state if the read indicates the known sequence in the target bin, and the probability to change to the minority fraction is f and the probability to change to the majority component is 1−f; and
presenting on a display, output data that indicates a condition for the minority component.

6. The system as recited in claim 5, wherein the one or more sequences of instructions further cause the apparatus to perform:
selecting a different candidate fraction f′ and determining the condition of the minority component for a different candidate fraction f′,
wherein said step of presenting the output data further comprises presenting output data that indicates the condition for the minority component based on the selected candidate fraction f or f′ for which the HMM better fits the amount data.

7. The system as recited in claim 5, wherein the amount data for the target bin is a weighted sum of amounts in the target bin and bins on either side of the target bin.

8. The system as recited in claim 7, wherein weights for the weighted sum are based on spreads of amounts in the target bin and of amounts in the bins on either side of the target bin among training data collected from a plurality of biological samples having only the majority component.
